(19) United States
(12) Patent Application Publication
(10) Pub. No.: US 2025/0127494 A1
(43) Pub. Date: Apr. 24, 2025

(54) STOOL COLLECTION AND EXTRACTION SYSTEM FOR HUMAN MICROBIOM ANALYSIS

(71) Applicant: Immundiagnostik AG, Bensheim (DE)

(72) Inventors: Franz Paul Armbruster, Bensheim (DE); Bernd Rittinghaus, Halver (DE); Ingmar Zude, Limburg-Offheim (DE)

(73) Assignee: Immundiagnostik AG, Bensheim (DE)

(21) Appl. No.: 18/685,728

(22) PCT Filed: Aug. 25, 2022
(86) PCT No.: PCT/EP2022/073728

§ 371 Date: Feb. 22, 2024

(30) Foreign Application Priority Data
Aug. 27, 2021 (DE) 10 2021 122 302.4

(51) Int. Cl. A61B 10/00; C12Q 1/6806

(52) U.S. Cl. CPC A61B 10/0038; C12Q 1/6806

(57) ABSTRACT

A specimen collecting system (10) with an elongated sampling utensil (100) for fecal matter and an integrated container for transport, preservation, and extraction of the collected sample. The system comprises an open-ended tubular structure (30) with a fitting hole at an intermediate position (50). The tubular structure can be tightly closed at both ends by screw caps (20, 40). The top cap (20) is integrally molded with or attached to a sampling utensil (100) that is inserted into the tubular structure (30) in the axial direction. The bottom cap (40) forms a liquid-tight distal chamber (32) with the tubular structure (30) and the sampling utensil (100) when inserted into the fitting hole, which is, therefore, capable of receiving a buffer solution. The elongated sampling utensil (100) has a fenestrated segment (110) with a sampling cavity (104) at its axial distal end and a cut-out member (106) at its forward end. The outer dimensions of the sampling utensil and the fenestrated segment (110) cooperate with the fitting hole. The proximal compartment (33) of the tubular structure (30) contains integrally formed with the inner wall spaced guide ribs (310) which direct the sampling utensil (100) through the fitting hole so that fecal matter adhering to or exiting the fenestrated segment (110) is retained in the proximal compartment (33)

### TECHNICAL FIELD

The invention relates to a kit of parts for collecting, preserving, and transporting a specimen of fecal material to a clinical laboratory for analysis, particularly for human microbiome analysis (G01N 33/48, G01N 1/04, A61B 10/02, G01N 1/28).

### TECHNICAL BACKGROUND

There are numerous devices for sampling and handling fecal material and paste-like specimens, such as spatulas, scoops, ladles, spoons, slot samplers, lancer samplers, or dippers. The sampling systems for occult blood testing typically consist of a screw cap connected to a rod or a spoon and a tube prefilled with a preservation buffer. For taking a fecal sample, the rod is targeted into the fecal material and inserted through a fitting hole into the chamber with the extraction buffer. The extraction buffer will keep the fecal specimen wet and preserve reagents mixed in addition to that slow down the biological degradation of the analyte. The amount of specimen picked by a grooved rod is small and variable. Alternatively, the fecal material may be weighed in and/or shuffled into the sampling tube using a spoon (cf. EP 1 384 442A1, U.S. Pat. Nos. 5,246,669 A, 5,514,341 A).

DE 10 2008 057866 A1 discloses a sampling device comprising a vessel, a sampling rod with shaped recesses, and a cap part containing an insertion hole with a tapered wiper sleeve. When the sampling rod is passed through the insertion hole into the vessel filled with buffer, the sleeve strips off any excess stool from the rod. The rod also serves as a plug for the insertion hole. DE 10 2012 109457 B4 describes an improved sampling system comprising a tubular vessel with buffer and a plug head with a tapered insertion portion and a specific outer periphery for avoiding leaking of the vessel. EP 3629017 A1 describes a manipulation-safe sampling device where the stool collection rod cannot be withdrawn once inserted through the insertion hole.

EP 1 366 715 B1 discloses a sampling device comprising a buffer-filled tubular container that is open at the top end and wherein a top closure member is provided with a threaded rod, and when the closure is fitted into the top end, the rod projects in the axial direction into the interior of the tubular container. The tubular container has a partition at an intermediate position which divides the container into a proximal compartment and a distal chamber. The partition is provided with an aperture that allows an axial passage of the threaded rod and any sample contained in its recesses but retains any excess stool in the said upper chamber. The bottom opening of the tubular container is provided with a lower closure member so that said device can be used as a sampling tube and placed into a sample holding plate of an automatic analyzer unit. For automated processing purposes, the bottom closure member may have a blind hole, e.g., having a hexagonal shape so that it can be removed automatically from the tube. This collection system is disadvantageous because a threaded rod only has small recesses and can only be used to collect small amounts of fecal material. In addition, when the rod is passed through the aperture, some grooves and recesses will become emptied because of the viscosity of the stool. The amount of fecal material transferred into the extraction buffer is too variable for diagnostic purposes and accurate quantitative determinations. The prior art represents a problem.

### BRIEF DESCRIPTION OF THE INVENTION

According to the invention, the problem is solved by a specimen collecting system, comprising:
(i) a tubular structure (30) open at the top and bottom ends (35, 38), wherein there is a structured partition with a fitting hole at an intermediate position (50) dividing the tubular structure (30) into a proximal compartment (33) and a distal chamber (32), and which is closable at both ends by a top cap and a bottom cap (20, 40), respectively;
(ii) a top cap (20) integral with or attached to a sampling utensil (100) that can be inserted in an axial direction into the tubular structure (30) and through the fitting hole into the distal chamber (32) when the top cap (20) is threaded onto the top end of the tubular structure (30);
(iii) a bottom cap (40) which, with the tubular structure (30), the structured partition (300) at the intermediate position (50), and the sampling utensil (100), when inserted axially into the fitting hole, forms a liquid-tight distal chamber (32) capable of containing a buffer solution; characterized by:
(iv) an elongated sampling utensil (100) which is tubular shaped and transitions at its axial distal end a fenestrated segment (110) that has a defined sampling cavity (104) and at its front end a cut-out member (106), the external dimensions of the tubular shaped part (102) and the fenestrated segment (110) being essentially the same as the dimensions of the fitting hole of the partition; and in that
(v) the proximal compartment (33) contains integrally with the inner wall of the tubular structure (30) and in axial direction located before the intermediate position (50) spaced guide ribs (310) which direct the fenestrated segment (110) of the elongated sampling utensil (100) through the fitting hole so that any specimen adhering to the elongate part (102) of the sampling utensil (100) or exiting the fenestrated segment (110) is retained in the proximal compartment (33) and displaced into the free space between the guide ribs (310) or any other clearance provided by the proximal compartment (33).

In some embodiments, the specimen collecting system comprises:
a transparent tube divided into a proximal compartment and a distal chamber and having a fitting hole at an intermediate position;
a screw cap attached to or integrally formed with an elongated sampling utensil (100) extends in the axial direction into the tube when the screw cap is screwed onto the top opening;
a screw cap at the bottom tube opening which, together with the tube, the partition, and the elongate sampling utensil, forms a liquid-tight distal chamber capable of receiving an aqueous buffer; wherein:
the elongated sampling utensil has a fenestrated segment (110) at its distal end that also provides a defined sampling cavity (104) and a cut-out member whose dimensions define the desired sampling volume, preferably from 50 to 500 microliters, more preferably from 50 to 250 microliters, and most preferably from 80 to 150 microliters;
the dimension of the fitting hole matches the dimensions of both the elongated portion (102) and the fenestrated segment (110) of the sampling utensil (100) such that a slidable liquid-tight seal is obtained at the fitting hole against the distal chamber when the sampling utensil is inserted; and
the proximal compartment includes integral spaced guide ribs (310) that direct the fenestrated segment and the elongated part of the sampling utensil (100) to and through the fitting hole that retains any fecal material adhering to the elongated part or exiting the fenestrated segment through its lateral openings (112) and/or slits and that the central portion of the sampling utensil displaces the retained or excess fecal material into the space formed by the spaced guide ribs or into the space or clearance between the central portion of the sampling utensil and the inner walls of the proximal compartment.

The elongated sampling utensil may be attached to the top screw cap via an attachment ring (290) such that it can slightly wobble, vibrate, and rock at its distal end when the entire specimen collecting system (10) is swirled, shaken, or rocked. This greatly facilitates the release of the pasty fecal material from the sampling cavity (104) through the lateral openings (112) and the opening at the front end so that the fecal matrix is extracted and/or dispersed solubilized in the buffer pre-filled in the distal chamber. The fenestrated segment (110) containing the sampling cavity (104) may comprise a series of structural elements with defined smooth, beveled surfaces so that the excised sample volume can pass into the buffer. The main advantage of the specimen collecting system disclosed in the present application is that a defined fecal sample of about 100 micrograms or more can be obtained reproducibly in a hygienic manner and in particular without weighing or increased visual or olfactory exposure to the feces, which is immediately sealed airtight in a container and extracted in a suitable preservation buffer so that analysis can subsequently take place. The specimen collecting system further allows the handling and processing the extracted fecal sample in an automated analysis unit. Conventional rod-based collection tubes for occult blood analysis are less accurate in sample volume and limited to samples of a few tens of micrograms.

Another aspect of the invention concerns the use of the specimen collecting system (10), which is particularly convenient since the elongated part of the sampling utensil (100) is inserted into the fitting hole up to position “1” only before sample collection, but in which a completely liquid-tight seal against the distal chamber is already provided. The distal chamber (32) can contain a liquid, e.g., an aqueous sample extraction buffer. From position “1,” the user can effortlessly unscrew the top cap (20), to which the sampling utensil (100) is attached, and pull it out upwardly, holding the tube body vertically in hand. The user can then place the opened tube system (10, 30) upright on a flat surface using the bottom screw with the placement ring. The user can then collect a fecal sample by dipping the elongated distal part of the sampling utensil with the fenestrated segment (110) into the stool. The elongated part with the fenestrated segment is reinserted into the tubular structure and passed through the fitting hole into the distal chamber with the buffer. The top screw cap (20) is then screwed on tightly with the utensil until an audible click is heard in position “2” so that the top cap (20) is secured against unintentional loosening or slipping off and, in addition, a completely liquid-tight seal is again provided at the fitting hole against the distal chamber (32). When the sampling utensil (100) is reinserted into the tube body and guided through the fitting hole, fecal material adhering to the elongated portion (102) or emerging from the lateral openings (112) of the fenestrated segment (110) is wiped and scraped off and displaced into the free space between the guide rips (310) or the other free space within the proximal compartment (33). The specimen collecting system (10) of the invention has consequently different working positions “1” and “2”, one before and one after the collection of the fecal sample, so that the user can effortlessly collect a defined amount of fecal sample with the sampling utensil (100), by for example (i) pulling the sampling utensil out of the tube body and sticking or dipping it in the stool, and (ii) reinserting the elongated portion with the fenestrated segment back into the tube up to the distal chamber containing the buffer and firmly screwing on the top cap until an audible click of the latch is heard. The sampling utensil with the shaped fenestrated segment, in combination with the fitting hole, defines a pre-defined volume of dense sample that is transferred into the buffer of the distal chamber. Weighing of the sample is not required, and olfactory exposure is minimized. No buffer or fecal sample is spilled as the system can be held vertically in hand and/or placed upright on a desk, shelf, rack, or tray.

Thus, in some preferred embodiments, the top cap may be a screw cap in which the internal thread includes a projection that engages or snaps on a detent (28) or latching nose at the end of the thread (22) outside the top opening of the tubular structure. The combination of latching nose or detent (28) and stop rim (34) provides an irreversible closure of the upper screw cap (20). It allows using similar-handed external threads (22, 44) at the top and bottom ends for manual opening and compatibility with tube capping and decapping systems increasingly used in the medical and laboratory industry. The snap or latch mechanism in the top screw cap (29), in conjunction with the stop rim (34), therefore, provides a reliable irreversible closure of the top screw cap (20). It prevents accidental loosening or opening by entrainment when the bottom closure cap (40) is removed or unscrewed to process the sample. Therefore, the specimen collection system is suitable for both manual handling and automated processing systems, as there is no risk of accidentally leaking.

In some embodiments, the sampling utensil (100) comprises a distal elongated portion (102) whose dimensions match the dimensions of the fitting hole and a central portion (200) whose sizes are larger so that the resulting transition section displaces aside retained fecal material and eventually takes a seat on the guide ribs (310) to the fitting hole.

In some embodiments, the sampling utensil (100) is shaped to include a central portion sized to take a seat on the spaced guide ribs (310) integrally molded with the inner tube wall and tapering to an annular fitting hole at an intermediate position (50); (ii) in that the central portion transitions distally to an elongated portion sized for sliding and sealing engagement with the annular fitting hole, and, when inserted, extends with the fenestrated segment (110) into the distal chamber (32; and (iii) in that proximal the central portion is a portion (200) sized for engagement with the inner wall of the tube body proximate its top opening (35). This embodiment offers the advantage that the proximal compartment of the tubular system is olfactorily sealed inside the tubular structure and externally sealed by the screw cap threaded onto the top opening (35).

In some embodiments, the specimen collecting system includes a distal chamber (32) pre-filled with a buffer or liquid for preservation, solubilization, and/or extraction of a pasty matrix. For example, the partially assembled system can be pre-filled through the bottom opening (35) after inverting and is then completely sealed by the bottom cap (40) for shipping and use. The free volume of the distal chamber (32) can be from 0.5 to 25 mL, preferably from 1 to 5 mL. The specimen collecting system (10), according to the invention, can be used to collect pasty samples of all kinds, which can be excised using a cup-or cylinder-shaped sample cutter with “ventilation openings,” e.g., a fenestrated hollow segment with a defined internal volume and a cut-out member (116) at its front end. The fenestrated segment preferably has beveled sidewalls and correspondingly inclined surfaces on the ceiling and around the openings as well as on the cutter member, all of which are optimized to remove a defined volume of the pasty sample.

The main application of the specimen collecting system of the invention is for the collection of a human stool sample, e.g., for PCR analysis of the microbiome or assays for viral or microbial nucleic acids, e.g., Rotavirus, Norovirus, Adenovirus, SARS, Norwalk pathogen, Semliki Forest Virus, or any other virus or bacterial nucleic acids of E. coli, Salmonella, Shigella, S.typhi, Paratyphi, Campylobacter, Clostridium, Staphylococcus, etc. or nucleic acids from parasites like Giardia, Entamoeba, Ascaris, etc. The advantage is that the sample size is not limited to 15 to 20 micrograms, as is the case with conventional devices for testing stool for occult blood using a rod with recesses. The specimen taken can have a specific pre-defined volume as needed, ranging from 50 to 500 micrograms, weight per volume. The specimen volume is preferably between 50 to 250 microliters, and particularly preferably between 80 and 150 microliters, e.g., if the specimen or stool has a jelly-like or pasty consistency. After the sample is cut out and removed, excess sample adhering to or leaking from lateral “vents” of the distal segment is scraped off at the intermediate position through the fitting hole so that the sample volume and its density are further adjusted and defined. This allows a quantitative analysis of the target analytes in the sample.

In some embodiments, the bottom screw cap has a nose that snaps over a stop, latch, or detent when tightly screwed onto the bottom opening. The bottom cap is then secured against unintentional removal or loosening.

In some embodiments, the external threads and screw caps for the top and bottom openings are left-and right-handed and must be rotated in opposite directions to open or close. This prevents misuse or incorrect rotations, particularly in fully automated analysis units. Alternatively, the detents or latches are such that the specimen collecting system can be used manually and in automated analysis systems. In some of these embodiments, an audible click is heard when the screw caps are in a locked position. In this preferred case, the opposing threads and caps may be identically threaded.

In some embodiments, at least one screw cap may include a molded blind hole or bushing, which cooperates with an automated opening tool (not shown). The bushing may have an internal polygon or a specific structure selected from tri-angle, Robertson, hex socket, security hex, double square, triple-square, 12-spline flange, double hex, pentalobular, hexalobular, Torx, security Torx, Torx plus, polydrive, Torx ttap, external Torx, line head male, line head female, line head female tamper; Trident (trident-point, trident groove, trident wing), Special (clutch A, clutch G, one-way, Bristol, Quadrex, Pig nose).

In some other embodiments, the screw caps may be provided with engagement and alignment means that fit or are suitable for mechanical bushing. This is particularly useful when the collection system is used with a rack in an automated analysis unit. In some more embodiments, the screw caps may also be provided with structured bushings suitable for opening tools, as mentioned above.

In some embodiments, at least one screw cap (40) can include a stand-up (set-up or placement) ring at the face end so that the collection system can be easily placed upright onto a flat surface, bench, shelf, tray, or table.

In some embodiments, the tubular structure (30) of the specimen collecting system (10) is molded with an axially elongated fitting hole provided with axially spaced annular sealing beads or sealing lips to ensure a liquid-tight seal against the distal chamber (32). Alternatively, the sealing beads may be the applied reverse on the elongated portion (102) of the sampling utensil (100).

In some embodiments, the specimen collection system includes additional sealing elements, sealing beads, sealing strips, or sealing lips near the top and bottom openings of the tubular structure and within the screw caps as well as plugs, stoppers, diaphragms, membranes to minimize leakage, contamination, and smells.

Another aspect of the invention relates to a method for collecting a defined amount of fecal specimen comprising the use of a specimen collecting system as described above and the following steps:—(i) pressing, indenting or dipping a fenestrated segment (110) of an elongated sampling utensil (100) into hard, doughy, sticky, plastic, or pasty feces to cut out a specimen like a cookie and collect a defined volume of fecal specimen, and (ii) inserting the portion with the fenestrated segment guided axially through a fitting hole by spaced ribs to wipe and remove any excess volume of fecal specimen—for example, amounts in exceeding the defined sampling capacity of the hollow fenestrated segment and therefore discharging from its outlets (vents), or amounts of fecal matter adhering to the exterior of the elongated portion or fenestrated segment—to collect a defined amount of solid fecal specimen; and (iii) extracting, dissolving, and dispersing the defined volume of solid fecal matter in a buffer prefilled into the specimen collection system for analysis. The latter step optionally includes the preservation of one or more analytes and extracting the fecal matrix for quantitative analysis.

In some embodiments of the method, the collected fecal sample has a defined volume in the range from 50 to 500 microliters, preferably from 50 to 250 microliters, and most preferably from 80 to 150 microliters.

Another aspect of the invention relates to a method of collecting a defined amount of fecal specimens followed by a study of the microbial communities found in the fecal sample. In a preferred embodiment, the method comprises the steps of characterizing and analyzing the genetic composition of the human microbiome using PCR and an automated analysis unit. Next-generation sequencing (NGS) has provided clinical laboratories with additional tools for metagenomics studies that allow surveying the genomes of entire microbial communities, including non-culturable microorganisms. However, this type of study requires larger fecal samples than can be collected with conventional rod-based systems, which have an upper sample volume of 20 microliters or micrograms. The weighing in of feces using a spatula or spoon cannot be automated and is, therefore, a significant barrier to introducing microbiome testing into the routine of a clinical laboratory.

The disclosed specimen collecting system is particularly advantageous because it allows the collection of defined sample volumes-regardless of whether the stool is pasty, doughy, hard, soft, slimy, or smeary-as the fecal matrix is immediately transferred to a buffer for preservation and extraction. Quantitative analysis of one or more biomarkers or a study of the microbiome requires a defined amount of sample of about 100 micrograms in a defined preservation and extraction buffer, which sample volume exceeds the volumes that can be collecting a sampling stick with recesses as in conventional sampling tubes for occult blood analysis. A sample weighing is impracticable and cannot be left to the patient. The disclosed specimen collecting system is self-explanatory and easy to use, allowing patients to handle it themselves. The specimen collecting system is suited for an automated analysis unit as the tube can be inverted, and the solubilized or extracted specimen be accessed from the top. If the tube has a septum at the bottom end, the sample fluid can be accessed from below using a needle. Therefore, the specimen collecting system is suitable for most automated analysis units for microbiome analysis.

Further features and preferred alternatives are disclosed in the claims and sub-claims. Additional advantageous objects and features appear from the following detailed description of a preferred embodiment with reference to the drawings.

### BRIEF DESCRIPTION OF THE DRAWINGS

It is shown in:

FIG. 1 a schematic drawing of the essential parts of the specimen collecting system according to the invention, comprising a top cap (20) attached to an elongated sampling utensil (100), a tubular structure (30), and a bottom cap (40);

FIG. 2 a perspective top view and a schematic drawing of the assembled specimen collecting system (10) of FIG. 1 before usage, wherein the sampling utensil (100) is axially inserted into the tubular structure (30) to position 1;

FIG. 3 a perspective bottom view and a schematic drawing of the assembled specimen collecting system (10) of FIG. 1 after usage, wherein the sampling utensil (100) is inserted into the tubular structure (30) to position 2;

FIG. 4 perspective rear and plan views of the sampling utensil (100), its distal elongated portion with the fenestrated segment (110) and the proximal portion (280);

FIG. 5 a schematic drawing of the sampling utensil (100) with a detailed view of the fenestrated segment (110) for collecting a sample in a sampling cavity (104);

FIG. 6 a cross-sectional drawing of the sampling utensil (100) with the fenestrated segment (110) of FIG. 5;

FIG. 7 a drawing of detail B of FIG. 6 and the fenestrated segment (110) with a sampling cavity (104);

FIG. 8 a schematic drawing showing in the upper part the sampling utensil (100) with the top screw cap and in the lower part the same sampling utensil (100) axially inserted into the tubular structure (30);

FIG. 9 cross-sectional drawings showing the specimen collection system (10) and the sampling utensil (100) assembled in position “1” in the upper part and position “2” in the lower part;

FIG. 10 a cross-sectional view of the fitting hole (300) within the tubular structure (30) and with a sampling utensil (100) inserted to position “2”;

FIG. 11 a cross-sectional drawing of the tubular structure (30) in the upper part and a detailed representation of the portion with the fitting hole in the lower part;

FIG. 12 a detailed cross-sectional drawing of the top screw cap (20) as well as of the top portion of the tubular structure (30) and the proximal portion of the sampling utensil (100) in position “1”;

FIG. 13 is a detailed perspective drawing showing, in the upper part, the bottom screw cap (40) with the placement ring and bushing and, in the lower part, a detailed cross-sectional drawing of the same screw cap (4) when screwed on the tubular structure.

### DESCRIPTION OF PREFERRED EMBODIMENTS ACCORDING TO THE INVENTION

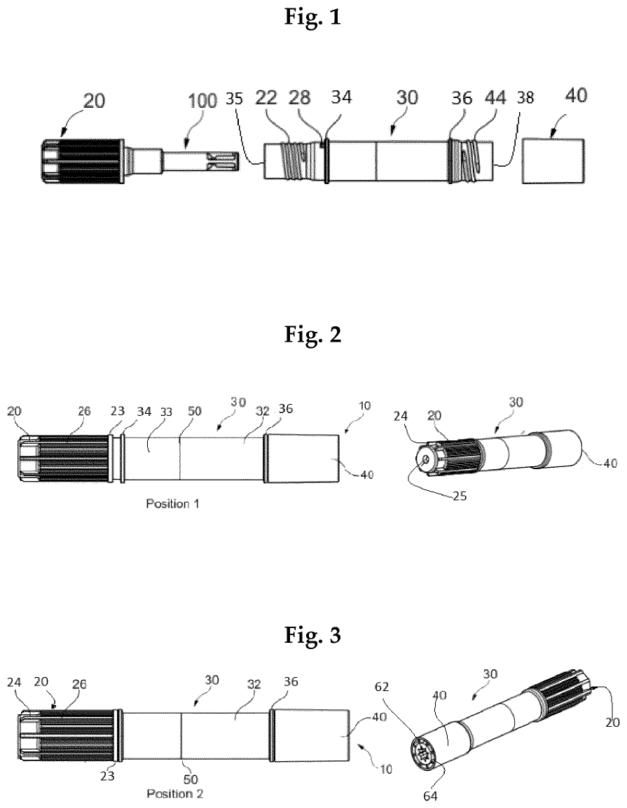

FIG. 1 shows the major components of a specimen collection system 10 for taking a pasty sample and transferring a predetermined volume of that pasty sample to a predetermined amount of a buffer for extraction and preservation of analytes. The analysis may be a characterization of the human fecal microbiome, illustrating the need for a well-defined sample size. As shown in FIG. 1, the specimen collection system 10 according to the invention, comprises a top closure cap 20 connected to a sampling utensil 100, a tubular structure 30 open on both sides, and a bottom closure cap 40. The top closure cap 20 and the sampling utensil 100 are integrally connected to each other. The tubular structure 30 is provided with external threads 22, 44 in the region of the top and bottom openings 35, 38 so that the openings can be closed by suitable screw caps 20, 40. The threads 22 44 may each have detents and 28, 29 so that an audible click is heard when the screw cap 20, 40 is securely and tightly screwed onto the opening 35, 38. External stop rims 34, 36, possibly with fillets, can be provided on the tubular structure 30, which cooperate with a stop ring 23 on the screw cap 20, 40 so that the tubular structure 30 is additionally sealed when the screw cap has been firmly screwed on.

FIG. 2 shows an assembled system 10 with the sampling utensil 100 inserted axially into the tubular structure 30 at position “1,” approximately four-fifths to nine-tenths. A perspective plan view of the assembled system 10 is shown on the right-hand side of the figure. The top closure cap 20 may further include means for alignment and positioning, such as a blind alignment hole 25 in the top base, and engagement means 24, such as ribs, that may cooperate with a corresponding rack socket (not shown). This allows the processing of specimen collecting system 10 in an automated analysis unit (not shown). The top closure cap 20 also has knurled grooves 26 for ease of manual handling. The stop ring 23 of the top closure cap 20 is still spaced from the top stop rim 34 in position “1”. The tubular structure 30 is divided into a distal chamber 32 and a proximal compartment 33 at the intermediate position 50. The distal chamber 32 of an assembled system 10 can receive a defined amount of liquid, e.g., a suitable extraction and preservation buffer since the fitting hole within the partition at the intermediate position 50 is closed in position “1” in a liquid-tight manner by the inserted elongated part 102 of the sampling utensil 100. The buffer chamber 32 of the tubular structure 30 is closed at the open end 38 by the closure cap 40 when the system 10 has been prefilled with buffer or when a sample is taken using the sampling utensil. System 10 may be prefilled with a defined amount of buffer for extraction and preservation of the sample through the bottom opening by turning the assembled system 10 upside down (overhead), i.e., after the elongated part of the utensil 100 has been inserted into the fitting hole at the intermediate position 50 in a fluid-tight manner. In use, the bottom opening of the tubular structure 30 is closed by a screw cap 40, but for automated prefilling or sample analysis, the bottom screw cap 40 may optionally be provided with a socket 62 for a machine opening tool (not shown); cf. FIG. 3. The bottom closure cap 40 may also be provided at the base end with a ring 64 for the upright placing of the system. In FIG. 2, position “1” refers to a system 10 in which chamber 32 has been pre-filled with buffer, and the sampling utensil 100 has been used only to seal the through-hole at the intermediate position 50 but not yet to take a sample of a pasty specimen. Since the top closure cap 20 has not been completely screwed on to the detent 28 or arrest and the sampling utensil 100 has only been partially inserted into the fitting hole, the top closure cap 20 with the sampling utensil 100 can be pulled out of the system 10 by hand without much effort to take them and collect a sample. During this time, the opened system 10 can be safely placed upright on a flat table or shelf using the placement ring 64 on the bottom closure cap 40 so that the buffer remains in chamber 32. The bottom closure cap 64 preferably has a placement or storage ring, whereas the top closure cap 20 has a wobbly base around the alignment hole 25.

In the case of system 10 in position 2 of FIG. 3, the sampling utensil 100 has been used for sampling and then fully re-inserted into the tubular structure 30. The perspective view of FIG. 3 on the right part shows system 10 from below and an assembled collecting system 10 as intended for shipping the sample to the laboratory for analysis, i.e., after the sample has been extracted for preservation in the buffer of the distal chamber 32. The bottom screw cap 40 may be provided with a placement ring 64 so that the tube may be placed upright on a table or shelf, i.e., after it has been inverted. The bottom screw cap 40 has a socket 62 that can cooperate with an opening tool. A septum may be provided at the bottom opening so that the extracted sample fluid can be withdrawn using a needle. The socket may be of any kind, e.g., an internal polygon (tri-angel, Robertson, hex socket, security hex, double square, triple-square, 12-spline flange, double hex, hexalobular, Torx, security Torx, Torx Plus, polydrive, Torx® ttap®, external Torx, line head male, line head female, line head female tamper); pentalobular, tree-pointed (tri-point, tri groove, tri-wing), special (clutch A, clutch G, one-way, Bristol, Quadrex, pig nose). Various star-shaped twist locks can also be provided. The bottom screw cap 40 rests against the bottom stop rim 36 to seal the bottom opening 38 in a fluid-tight manner. The top screw cap 20 is screwed onto the top opening 35 in position “2” so that its stop ring 23 rests against the top stop rim 34. A detent 28 or latch may be provided at the end of thread 22 so that the top screw cap 20 is secured against accidental loosening. An audible click is heard when it has been screwed on tightly. Such a detent may also be present on the bottom thread 44. In the “2” position, the sampling utensil 100 is fully inserted in the axial direction into the fitting hole at the intermediate position 50 so that its elongated part 102 provides a tight seal between the distal buffer chamber 32 and the proximal compartment 33.

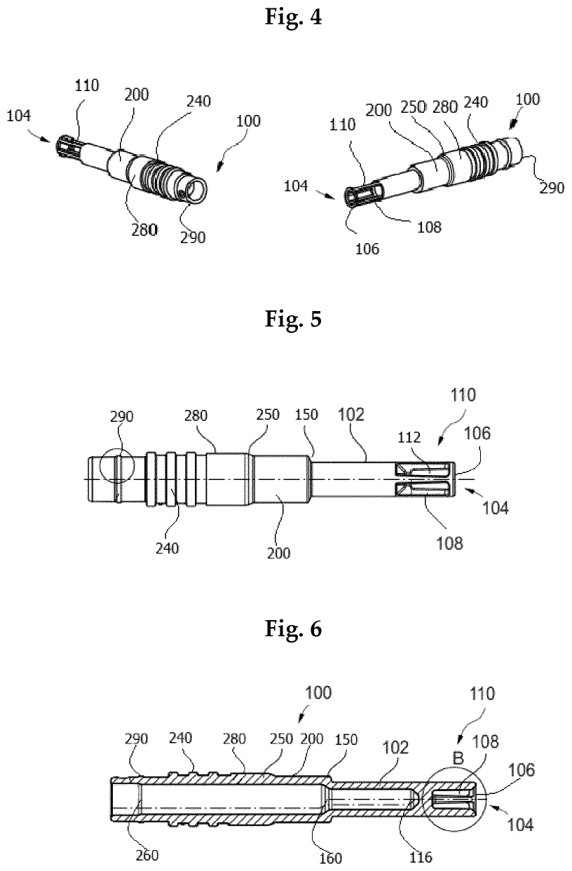

Referring to the top and rear views of FIG. 4, the sampling utensil 100 is injection molded and attached to the top screw cap 20 by a set of flange rings and U-profiles 240. Alternatively, the sampling utensil 100 may be integrally molded with the top screw cap 20. As shown in FIG. 4, the sampling utensil 100 comprises a distal elongated portion which transitions into a similarly sized fenestrated segment 110. Proximal to the long section is a larger central portion 200 and an even larger proximal portion 280, which transitions into a set of flange rings and U-profiles 240 for an interference fit. The transition section 150 is located between the elongated section 102 and central section 200. It is designed to rest in assembling position 2 on a set of guide rips and a fitting hole at the intermediate position 50. The other proximal portion 280 is sized to fit sealing against the inner walls of the top opening 35 of the tubular structure 30. The proximal portion 280 then transitions to a section with annular flanges and U-sections 240, allowing the sampling utensil 100 to be secured to the top cap 20. A terminal 290 sealing ring may be provided at the junction between the top cap 20 and the sampler 100. The sampling utensil 100 may be hollow, thus somewhat compliant. Fastening with ring flanges and profiles 240 between the cap 20 and the proximal part of the sampler 100 allows fixed or loose fastening, depending on the dimensions used.

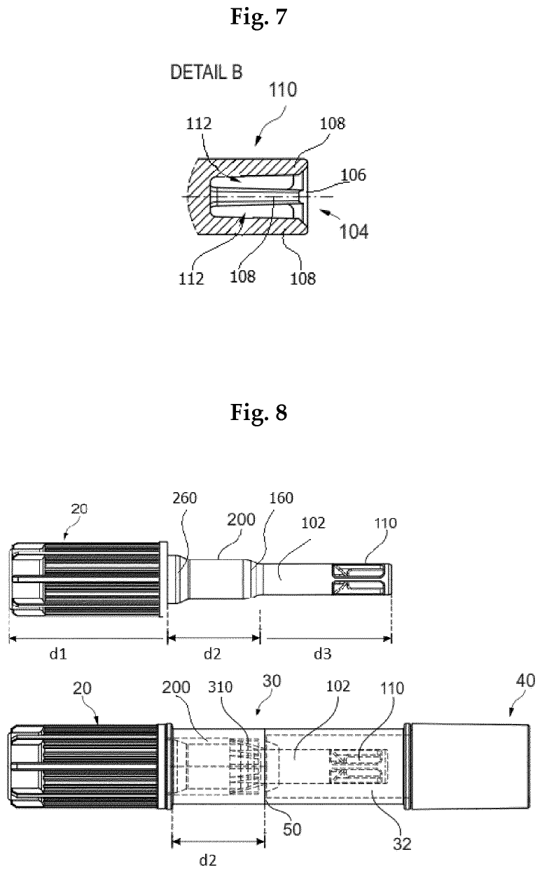

FIG. 5 shows a side view of the fecal matter collection utensil 100 of FIG. 4, and FIG. 6 further shows a cross-sectional drawing. The proximal section 280 corresponds to the inner diameter of the tubular structure 30, so a specific interference fit (press fit) is to be achieved. The fecal matter collection utensil 100 tapers distally in a transition section 250 to a central section 200, the outer diameter of which is smaller than the inner diameter of the tubular structure 30, so that there is a clearance 320 for excess fecal matter to be scraped from the elongated section 102 or the fitting holes 112 of the fenestrated basket-like segment 110 of the sampling utensil 100. The diameter of the central portion 200 then tapers distally in a transition section 150 into the elongated part 102, which transitions into a fenestrated basket-shaped segment 110 having a cutout-shaped member 106 at its forward end. FIG. 7 is a drawing of detail B of FIG. 6. The fenestrated basket-shaped segment 110 has an (annular) cutout member 106 supported by (usually three or four) beveled structural elements 114. These are beveled and shaped to allow excess collected fecal material to exit the sampling cavity 104 of the fenestrated basket-like segment 110 with little resistance. This prevents the sample from being excessively compacted when the sampling utensil 100 is inserted into the tubular structure and forcefully pushed through the fitting hole 52 in the partition wall 50 of the tubular structure 30. The volume of the sampling cavity 104 can be adapted to individual requirements. A sample volume of 100 microliters corresponds to an average of 100 micrograms of feces and is too large to be picked up by conventional sampling rods with defined grooves and indentations. The surfaces of the elongated part 102 are smooth so that adhering feces can be scraped or wiped off easily. The fecal sample is collected by first pressing the fenestrated basket-like segment 110 into the pasty feces with the cutter 106, thus cutting out a defined sample volume like a cookie. The sample volume is limited by the volume of the sampling cavity 104 of the fenestrated segment 110. Subsequently, when the elongated part 102 with the fenestrated segment 110 is pushed through the fitting hole 52, any fecal matter adhering to the outside of the elongated part 102 and/or exiting the lateral openings 112 of the sampling cavity 104 is retained in the proximal compartment 33, which has free spaces for excess fecal material between the guide ribs 310 and between the central part 200 and the inner wall of the tubular structure 30. Therefore, the lateral openings 112 of the fenestrated segment 110 ensure that the sampling cavity 104 is uniformly filled with pasty sample and that it is not further compacted or loosened. The fenestrated segment 110 has three to five structural elements 108 with beveled sides, allowing the pasty sample to exit into the distal chamber buffer when the system is gently shaken or agitated. The goal is to have fecal samples of equal volume that are as uniformly dense as possible, which are then solubilized and extracted in the predetermined buffer volume of the distal chamber. This chamber is preferably filled with a 1.5 ml extraction and preservation solution. In some embodiments, the sampling utensil 100 is loosely or rotatably connected to the top closure cap 20 so that the pasty sample is additionally forced out of the sampling cavity 104 through the lateral openings 112 and actively dispersed in the buffer of the distal chamber 33 due to the greater vibrations once the system 10 is swirled or shaken, for example.

FIG. 8 shows, in the upper part of the figure, the sampling utensil 100 mounted with the top screw cap 20 in the proximal region (d1). The central region (d2) and the distal region (d3) of the sampling utensil 100 are inserted in use into the tubular structure 30, which is closed by the bottom screw cap 40; cf. lower part of FIG. 6. The sampling utensil 100 is shown in a position where in the central region (d2) with the central portion 200 the transition 150 rests on the guide ribs 310 of the tubular structure 30 and the elongated section 102 of the sampling utensil 102 passes through the fitting hole in the partitioning wall 50. The elongated section 102 and the fenestrated basket-like segment 110 extend into the distal chamber, which is filled with a buffer for preservation and/or extraction of the fecal sample. The volume of the fecal matter excised by the fenestrated basket-like segment 110 at the distal forward end of the sampling utensil 100 is strictly defined by the dimensions of the sampling cavity. It may preferably be selected between 50 and 250 microliters, more preferably about 100 microliters. As the sample material is not compacted nor loosened, a very defined specimen volume can be drawn without weighing and immediately transferred to a buffer system to extract the fecal matrix and preserve the analyte.

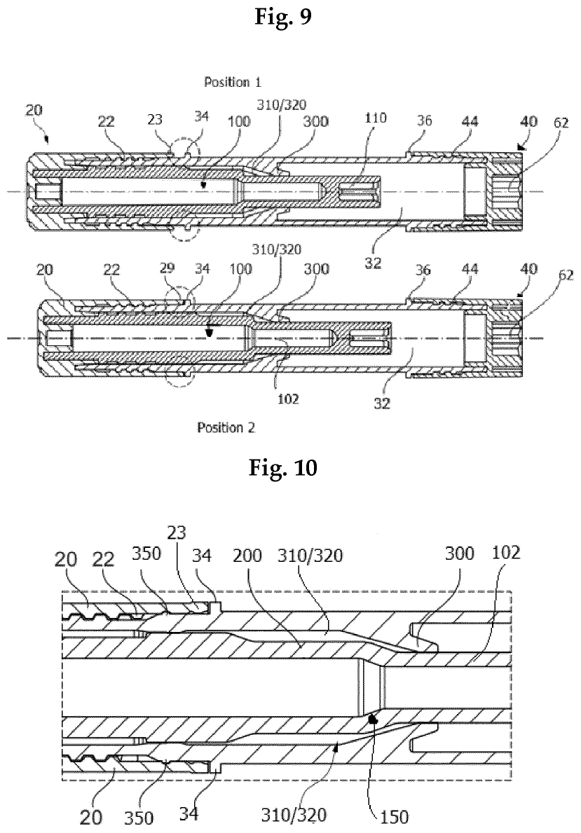

FIG. 9 shows two cross-sectional drawings of the sample collection system 10, wherein in the upper part of the illustration, the sampling utensil 100 is inserted into the tubular structure 30 only up to position 1, i.e., the top screw cap 20 is screwed four fifths to nine-tenths onto the external thread 22 of the tubular structure so that a gap remains between the stop ring 23 of the screw cap 20 and the stop rim 34 on the tube 30. The elongated part 102 of the utensil is stuck in the fitting hole 300 of the chamber separation so that no liquid can escape the extraction chamber 32. The fenestrated segment 110 is wetted by the liquid introduced into the distal extraction chamber 32. The distal chamber 32 is fully closed by the bottom screw cap 40; that is, the bottom screw cap is fully threaded onto the threads 44 at the bottom opening so that its stop ring contacts the bottom stop rim 36 on the tubular structure 30. The bottom screw cap 40 has a socket 62, so it can be automatically unscrewed using a special tool (not shown). In this position 1, the sampling utensil 100 can be removed manually from the stripping tube 30 with the aid of the screw cap effortlessly and without great force, e.g., for taking a pasty stool sample with the cutter at the front end. The lower part of FIG. 9 shows the sampling utensil 100 when it is axially reinserted into the tubular structure 30, now up to position 2, so that its fenestrated segment 10 and the entire elongated part 102 are axially reinserted through the through-hole 300 in the extraction compartment 32. The transition 150 rests on the guide ribs 310/320. Any excess paste-like stool that may have adhered to the elongated part 102 or exited through the lateral openings 112 of the sampling cavity 104 has been displaced into the spaces between the spaced guide ribs 310/320 and into the space formed between the central portion 200 and the inner wall of the tubular structure 30.

See FIG. 10, a detailed drawing of spaced guide ribs 310/320 merging into a fitting hole 300 within a partition separating the tubular structure 30 into a distal buffer chamber 32 and a proximal clearance compartment 33. The elongated part 102 of the utensil is dimensioned to seal the fitting hole 52 at position 352 within partition 300 in a fluid-tight manner. Sealing beads may be located on the elongated part 102 to sealingly correspond to axially different positions “1” and “2” of the inserted utensil 100; cf. FIGS. 2, 3 & 9

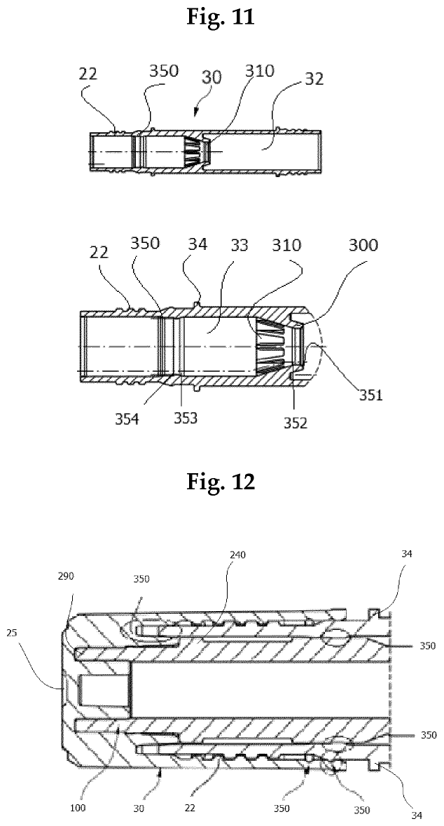

FIG. 11 is a schematic sectional drawing of the tubular structure 30 indicated by vertical lines 350, 351, 352, 353, and 354 locations of potential seals against distal buffer chamber 32 and proximal compartment 33. The upper part of FIG. 11 shows the location of the distal buffer chamber 32. The lower part of FIG. 11 is a detailed view of the guide ribs 310, which transition into a fitting hole in partition 300, where the excess sample is wiped off and retained from entering the distal chamber when the elongated sampling utensil (not shown in FIG. 11) is inserted into the tubular structure 30. The retained sample is displaced into the free space between the spaced guide ribs 310 and into the space formed by the clearance between the central region 200 of the sampling utensil 100 and the inner wall of the tubular structure 30. Seals 350, 354, and 353 are provided for odor-free transport and handling of the specimen collection system after sampling, in which “seals” 351 and 352 take the role of wiping away excess sample material. Sealing against liquid takes primarily place at positions “353” and “354” to ensure a liquid-tight buffer chamber 32 before and after sampling.

FIG. 12 is a sectional drawing of the various engagements between the top closure cap 20, to which the sampling utensil 100 is mounted internally, and the tubular structure 30 in the region from the top external thread 22 up to the stop rim 34. Also shown are the multiple abutments of the flange rings 240 of the sampling utensil 100 of the inner wall of the tubular structure 30 in the area where the closure cap 20 is screwed on the external thread 22, and the engagement of the fastening ring 290 of the sampling utensil 100 in a corresponding groove in the internal base of the closure cap 20. FIG. 12 further illustrates the numerous seal beads 350 and gaskets 350 between the tubular structure 30 and the sampling utensil 10 and between the closure cap 20 and the tubular structure 30.

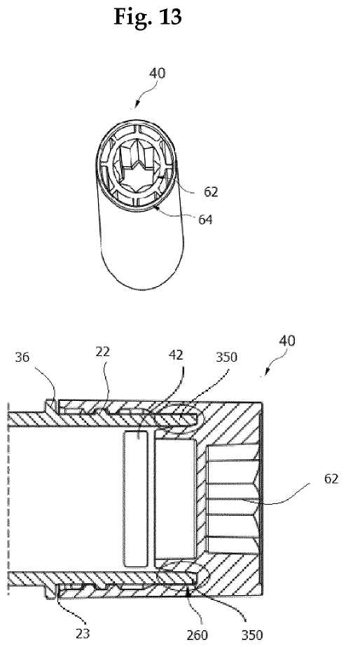

FIG. 13 is a detailed drawing of the bottom closure cap 40 and the various engagements between the tubular structure 30 and the closure cap. The opening of the tubular structure may optionally contain a septum, filter, or sieve 42, depending on the analytical needs. The closure cap 40 may have a stop ring for abutment of a bottom external rim for tight closure. A hexagonal socket 62 is shown, which cooperates with various machine tools. The external top and bottom threads 22 may have the same or different pitches and orientations (right and left-handed threads) to avoid accidental opening.

A person skilled in the art will appreciate that changes and variations of detail can be made to the described embodiments without departing from the representative example of a specimen collecting system outlined in the drawings and the description and from the gist of the invention.

### EXAMPLES

The specimen collection system was tested to determine whether it could be used for transport, preservation, and stabilization of feces (stool) and whether the extracted amounts of microbial DNA could be subjected to PCR analysis and sequencing. The amount of extraction buffer in the distal chamber was 2 mL, and the collected volume of pasty feces was 100 microliters.

It was found that the collected sample produced sufficiently high concentrations of nucleic acids in the nucleic acid eluates after isolation using the ZymoBiomics™ 96 MagBead DNA extraction system (ZymoResearch™, Freiburg Art. R2135). The concentrations obtained were in the range of 10 to 100 ng/u using the Quant IT™ Picogreen method (ThermoFisher Art. P7589), and the isolated microbial DNA could be used for qPCR analysis, e.g., to determine the ratios of Akkermansia, Bifidobacterium, Bacteroidetes in human stool. Four stool samples were collected from one stool of each patient, and different stools of patients were examined. The isolated amounts of DNA were analyzed by the Quant IT™ Picogreen method. The observed standard deviations of the quantities of isolated DNA over the respective stool samples were regularly lower than with other sampling methods, including weighing of stool.

The sampling method was further tested with pasty specimens spiked with Zymo Microbial Community Standard, which included microbial DNAs from Bacillus subtilis, Listeria monocytogenes, Staphylococcus aureus, Enterococcus faecalis, Lactobacillus fermentum, Salmonella enterica, Escherichia coli, Pseudomonas aeruginosa. The ratio of percentage target share to percentage actual share and the added deviation ratio was comparable to the pure standard, so the described specimen collecting system did not introduce an error due to the sampling method.

### SUMMARY

Therefore, the specimen collecting system of the invention allows a reproducible and effortless collection of human stool samples by the patient. The collected samples are large enough for microbiome studies based on isolated DNA and for target analytes lesser present in stool that needs to be purified and concentrated for their analysis. The sample size is larger and more defined than in rod-based sampling systems. The embodiments disclosed overcome the problem that a structured rod can have small recesses only and that some of these recesses will empty when passed through an orifice due to the pasty viscosity of some stool samples. In contrast, the fenestrated segment of the elongated sampling utensil described can be wider and accommodate a sampling volume of the required size. In addition, the forces exerted by the fitting hole on the sampled pasty stool are lower. Although the fitting hole must be larger in size, it can be closed by an elongated sampling utensil that is preferably hollow and slightly elastic so that it can seal liquid tight at the fitting hole. A skilled person will recognize that the plastic materials of the screw caps, the sampling utensil, and the tubular structure should have different hardness for fulfilling functions.

### Reference Signs

10 specimen collecting system
20 top screw cap
22 external threads
23 stop ring
24 engagement means
25 alignment hole
26 knurled grooves
28 detent (lock, latch, retainer)
30 open-ended tubular body (tubular structure)
32 distal buffer chamber (extraction chamber).
33 proximal clearance compartment (with free space for excess sample)
34 top stop rim
35 top opening (tubular structure)
36 bottom stop rim
38 bottom opening (tubular structure)
40 bottom closure cap
42 septum, filter, sieve
44 external threads (bottom cap)
50 intermediate position
52 through hole/fitting hole
62 socket-internal polygon
64 placement ring
100 sampling utensil (fecal matter collection utensil)
102 elongated part
104 sampling cavity
106 cut-out member (cutter)
108 beveled structural elements
110 fenestrated basket-like segment
112 lateral opening
150 transition section (seat on ribs)
200 central portion
250 top transition section
240 flange rings and U-profiles for abutment
280 proximal section
290 fastening ring/mounting ring
300 partition with through hole/fitting hole
310 spaced ribs/fins
320 clearance, free space (for removal of excess feces)
350 gasket/sealing bead
351 seal 1/position 2
352 seal 2/position 1
353 seal 3/position 2
354 seal 4/position 1
d1 proximal portion
d2 central portion
d3 distal section

1. A specimen collecting system with an elongated sampling utensil for fecal matter and an integrated vessel for extraction of the collected fecal sample, comprising:
(i) a tubular structure open at the top and bottom ends, wherein there is a fitting hole at an intermediate position dividing the tubular structure into a proximal compartment and a distal chamber, and which is closable at both ends by a top cap and a bottom cap, respectively;
(ii) a top cap integral with or attached to an elongated sampling utensil that can be inserted in the axial direction into the tubular structure and through the fitting hole into the distal chamber;
(iii) a bottom cap which, with the tubular structure and the elongated sampling utensil, when inserted axially into the fitting hole, forms a liquid-tight distal chamber capable of containing a buffer solution; characterized in that:
(iv) the elongated sampling utensil has at its axial distal end a fenestrated segment with a sampling cavity and at its forward end a cut-out member, the outer dimensions of which and the fenestrated segment cooperating with the dimensions of the fitting hole; and in that
(v) the proximal compartment contains integrally with the inner wall of the tubular structure and before the intermediate position spaced guide ribs which direct the fenestrated segment of the elongated sampling utensil through the fitting hole so that any specimen adhering to the elongated part of the sampling utensil or exiting the fenestrated segment is retained in the proximal compartment and displaced into the free space between the guide ribs or within the proximal compartment.

2. The specimen collecting system, as claimed in claim 1, wherein the elongated sampling utensil, when inserted into the tubular structure and engaged in a first position “1”, provides a semi-tight wiping seal at the fitting hole, and when inserted into the tubular structure and engaged in an axially forward second position “2”, provides a liquid-tight seal at the fitting hole.

3. The specimen collecting system as claimed in claim 2, wherein said top screw cap formed integrally with or connected to the elongated sampling utensil snaps in position “2” onto a detent arranged at the end of external threads near the top opening of the tubular structure.

4. The specimen collecting system as claimed in claim 1, wherein the sampling utensil comprises an elongated distal part whose outer dimensions match the dimensions of the fitting hole and a central portion whose larger dimensions are sized to take a seat on the spaced guide ribs that lead the elongated part to the fitting hole and to displace retained specimen into the free space between the guide ribs and within the proximal compartment.

5. The specimen collecting system as claimed in claim 1, wherein the fenestrated segment is provided with multiple lateral openings for an exit of excess sample material from the sampling cavity and at the open front end of the sampling cavity with a cut-out member.

6. The specimen collecting system as claimed in claim 1, wherein the sampling cavity of the sampling utensil provides a cut-out volume of 50 microliters to 250 microliters, preferably of 80 to 120 microliters, more preferably of 100 microliters.

7. The specimen collecting system as claimed in claim 1, wherein the elongated sampling utensil comprises
(i) a central portion dimensioned to seat on guide ribs integral with the inner wall of the tubular structure and located proximal to the fitting hole;
(ii) a distal elongated part sized for sliding and sealing engagement with the fitting hole and which projects axially with its fenestrated segment into the distal chamber when inserted; and
(iii) a proximal portion sized larger to abut the inner wall of the tubular structure at the top opening when inserted.

8. The specimen collecting system according to claim 1, any one of the preceding claims, wherein the distal chamber (33) is pre-filled with buffer for preservation, solubilization, and/or extraction of analytes of a fecal matrix.

9. The specimen collecting system according to claim 1, wherein the tubular structure is provided near the top and bottom end with external threads for screw caps and, when fully screwed on, that those engage with locking devices or latches for being secured against accidental or unintentional loosening or dislodgement.

10. The specimen collecting system according to claim 1, wherein the threads near the top and bottom ends are left-handed and right-handed to avoid unintentional opening of the other screw cap during handling.

11. The specimen collecting system according to claim 1, where an audible click is heard when a screw cap engages in a locking device.

12. The specimen collecting system according to claim 1, wherein the screw cap attached to the sampling utensil is knurled at its outside for better handling and provided with engagement and alignment means mating or suitable for a mechanical bushing or a positioning means.

13. The specimen collecting system according to claim 1, wherein at least one screw cap is provided with a receptacle capable of cooperating with an opening tool of an automated analyzer unit.

14. The specimen collecting system according to claim 1, wherein the tubular structure is provided with one or more stop rims, optionally with a fillet, for tight closure of the screw caps.

15. The specimen collecting system according to claim 1, wherein at least one screw cap has placement means for an upright placement of the tubular specimen collecting system on a flat surface.

16. The specimen collecting system according to claim 1, wherein the fitting hole within the partition is provided with axially spaced annular sealing elements such as sealing beads or sealing lips.

17. A method of collecting and extracting a defined amount of pasty fecal material which comprises the use of a specimen collecting system as disclosed in claim 1, comprising the steps of:
removal of the top screw cap with the elongated sampling utensil from the specimen collecting system and dipping the fenestrated segment of the sampling utensil into pasty fecal material to excise a volume of fecal sample, and
reinserting the elongated sampling utensil with the fenestrated segment in an axial direction through the fitting hole to obtain a defined volume of fecal material while wiping off and retaining in the proximal compartment any excess fecal material exiting the lateral openings of the fenestrated segment or adhering to the elongate part of the sampling utensil; and
extracting and solubilizing the excised volume of fecal matter in the sampling cavity in an aqueous buffer pre-filled into the distal buffer chamber of the specimen collecting system, which is now suitably shaken and agitated.

18. The method of collecting a defined volume of fecal matter as claimed in claim 17 for collecting a volume of a fecal sample of 100 microliters or 100 micrograms.

19. The method of collecting a defined amount of fecal specimen as claimed in claim 18, further comprising the steps of analyzing the patient's microbiome in an automated analysis unit.
